(19) United States
(12) Patent Application Publication
(10) Pub. No.: US 2026/0242565 A1
(43) Pub. Date: Aug. 20, 2026

(54) RESIN COMPOSITION

(71) Applicants: KANEKA CORPORATION, Osaka-shi (JP); NATIONAL UNIVERSITY CORPORATION KOBE UNIVERSITY, Kobe-shi (JP); NATIONAL INSTITUTE OF ADVANCED INDUSTRIAL SCIENCE AND TECHNOLOGY, Tokyo (JP)

(72) Inventors: Toshihiko KANDA, Takasago-shi (JP); Sho FURUTATE, Takasago-shi (JP); Shunsuke SATO, Takasago-shi (JP); Sangho KOH, Kobe-shi (JP); Seiichi TAGUCHI, Kobe-shi (JP); Yusuke IMAI, Tsukuba-shi (JP); Yuichi TOMINAGA, Tsukuba-shi (JP); Shinji TANAKA, Tsukuba-shi (JP); Masaru YOSHIDA, Tsukuba-shi (JP)

(73) Assignees: KANEKA CORPORATION, Osaka-shi (JP); NATIONAL UNIVERSITY CORPORATION KOBE UNIVERSITY, Kobe-shi (JP); NATIONAL INSTITUTE OF ADVANCED INDUSTRIAL SCIENCE AND TECHNOLOGY, Tokyo (JP)

(21) Appl. No.: 19/662,015

(22) Filed: Apr. 29, 2026

Related U.S. Application Data
(63) Continuation of application No. PCT/JP2024/039093, filed on Nov. 1, 2024.

(30) Foreign Application Priority Data
Nov. 1, 2023 (JP) 2023-187708
Nov. 1, 2023 (JP) 2023-187709

(51) Int. Cl. C08J 11/26

(52) U.S. Cl. CPC C08J 11/26

(57) ABSTRACT

A resin composition contains polylactic acid and a copolyester of lactic acid and another hydroxycarboxylic acid. The copolyester has a weight-average molecular weight of 11×10⁴ or more. The copolyester has a copolymerization randomness of 0.5 to 3.0, the copolymerization randomness being calculated as a ratio (b/a) of a theoretical value (b) of a triad content of the other hydroxycarboxylic acid to a measured value (a) of the triad content of the other hydroxycarboxylic acid. The copolyester of lactic acid and another hydroxycarboxylic acid can be used as a soil biodegradation accelerator for accelerating soil biodegradation of polylactic acid.

### REFERENCE TO A SEQUENCE LISTING

A Sequence Listing, submitted as an XML file and compliant with WIPO Standard ST.26, forms part of the present application. The Sequence Listing is identified as follows: File name “561843US_ST26.xml,” created on Apr. 16, 2026, with a size of 14,292 bytes.

### TECHNICAL FIELD

The present invention relates to a resin composition or a molded article containing polylactic acid, a soil biodegradation accelerator for accelerating soil biodegradation of polylactic acid, and the use of the soil biodegradation accelerator.

### BACKGROUND ART

Plastics are not readily degradable in the natural environment, and environmental pollution caused by the mass disposal of plastics has emerged as a serious problem.

To address this problem, biodegradable plastics that can be decomposed into water and carbon dioxide through the action of microorganisms have been increasingly put into practice.

Polylactic acid, which is a typical example of biodegradable plastics, is a biopolymer that can be synthesized from biomass-derived materials. Owing to its relatively high stiffness and strength and its transparency, polylactic acid has been increasingly used as a substitute for petroleum-based plastics in a wide range of applications.

However, polylactic acid is a highly rigid resin, and various techniques have been investigated to soften it. One known example of such softening techniques is to add a low-molecular-weight plasticizer such as isobutyl adipate, dioctyl sebacate, tributyl acetylcitrate, or triacetylene.

Another known technique is to blend polylactic acid with another polymer. For example, Patent Literature 1 describes blending polylactic acid with a copolyester of lactic acid and another hydroxycarboxylic acid, and states that such blending can plasticize the polylactic acid without substantially reducing its transparency.

It is also known that polylactic acid has a low biodegradation rate and insufficient biodegradability, particularly in soil. Polylactic acid cannot be biodegraded quickly by a common method such as home composting or soil burial, and remains in composts or soil for a long period of time. Thus, polylactic acid is not considered compostable.

Against this background, techniques for improving the biodegradability of resin materials containing polylactic acid have been investigated. For example, Patent Literature 2 discloses improving the biodegradability of a polylactic acid-containing material through the use of regenerated cellulose.

### CITATION LIST

### Patent Literature

PTL 1: WO 2020/066679 A1
PTL 2: WO 2022/085725 A1

### SUMMARY OF INVENTION

### Technical Problem

According to the technique described in Patent Literature 1, polylactic acid can be softened. However, the blend may lack sufficient melt viscosity or melt tension during melt processing. Accordingly, drawdown immediately after extrusion of the melt from an extruder is likely to be excessive, which can lead to reduced workability or molding defects.

Although Patent Literature 2 discloses a technique for improving the biodegradability of a polylactic acid-containing material, the improvement effect is insufficient. In addition, mixing polylactic acid with a substance having a markedly different chemical structure entails the risk of impairing the transparency or mechanical properties of the polylactic acid.

In view of the above circumstances, a first aspect of the present invention aims to provide a polylactic acid-containing resin composition that exhibits improved tensile elongation at break of polylactic acid and reduced drawdown during melting.

A second aspect of the present invention aims to provide a novel technique for accelerating soil biodegradation of polylactic acid.

### Solution to Problem

The present inventors have found that blending polylactic acid with a copolyester of lactic acid and another hydroxycarboxylic acid that has specific features can improve the tensile elongation at break of the polylactic acid and reduce drawdown during melting. Based on this finding, the inventors have arrived at the first aspect of the present invention.

The present inventors have also found that a copolyester of lactic acid and another hydroxycarboxylic acid can accelerate soil biodegradation of polylactic acid. Based on this finding, the inventors have arrived at the second aspect of the present invention.

Specifically, the first aspect of the present invention relates to a resin composition containing:
polylactic acid; and
a copolyester of lactic acid and another hydroxycarboxylic acid, wherein
the copolyester has a weight-average molecular weight of 11×10⁴ or more, and
the copolyester has a copolymerization randomness of 0.5 to 3.0, the copolymerization randomness being calculated as a ratio (b/a) of a theoretical value (b) of a triad content of the other hydroxycarboxylic acid to a measured value (a) of the triad content of the other hydroxycarboxylic acid.

The first aspect of the present invention also relates to a molded article produced by molding the resin composition.

The second aspect of the present invention relates to a soil biodegradation accelerator for accelerating soil biodegradation of polylactic acid, the soil biodegradation accelerator containing a copolyester of lactic acid and another hydroxycarboxylic acid.

The second aspect of the present invention also relates to a resin composition containing polylactic acid and the soil biodegradation accelerator and to a molded article produced by molding the composition.

The second aspect of the present invention further relates to a method for accelerating soil biodegradation of polylactic acid, the method including bringing a copolyester of lactic acid and another hydroxycarboxylic acid into contact with the polylactic acid.

The second aspect of the present invention further relates to use of a copolyester of lactic acid and another hydroxycarboxylic acid as a soil biodegradation accelerator for accelerating soil biodegradation of polylactic acid.

### Advantageous Effects of Invention

The first aspect of the present invention can provide a polylactic acid-containing resin composition that exhibits improved tensile elongation at break of polylactic acid and reduced drawdown during melting. As a result, it is possible to improve the tensile elongation at break of a molded article containing polylactic acid. In addition, when the polylactic acid-containing resin composition is subjected to melt processing, a reduction in workability and the occurrence of molding defects can be suppressed.

The second aspect of the present invention can provide a novel technique for accelerating soil biodegradation of polylactic acid. According to a preferred embodiment, soil biodegradation of polylactic acid can be accelerated without reducing the biomass level of the polylactic acid.

According to a preferred embodiment of the second aspect of the present invention, soil biodegradation of polylactic acid can be accelerated without substantially reducing the transparency of the polylactic acid. Furthermore, the polylactic acid can be plasticized to increase elongation.

### BRIEF DESCRIPTION OF DRAWINGS

FIG. 1 shows an example of a chart of a ¹H-NMR spectrum measured for a copolyester of lactic acid and 3-hydroxybutyric acid, and is an enlarged view of a region around 1.3 ppm in the spectrum.

FIG. 2 is a scanning electron microscope image captured by observing a freeze-fractured surface of a press sheet of Example 8 which was formed from polylactic acid and a copolyester.

FIG. 3 is a scanning electron microscope image captured by observing a freeze-fractured surface of a press sheet of Comparative Example 9 which was formed from polylactic acid alone.

FIG. 4 is a histogram showing a diameter distribution measured for 200 circular concave features in the scanning electron microscope image of FIG. 2.

### DESCRIPTION OF EMBODIMENTS

Hereinafter, embodiments of the present invention will be described.

### [First Aspect]

The resin composition according to the first aspect of the present invention contains polylactic acid and a copolyester of lactic acid and another hydroxycarboxylic acid.

### (Polylactic Acid)

The polylactic acid is a polyester containing lactic acid as a constituent monomer. The polylactic acid may be conventionally known polylactic acid and may be either crystalline or amorphous. The polylactic acid is preferably a homopolymer of lactic acid, but may contain a small amount of another monomer in addition to lactic acid.

The lactic acid of the polylactic acid may be either L-lactic acid or D-lactic acid or may be a combination of both. In the latter case, the ratio between L-lactic acid and D-lactic acid is not limited to a particular range.

The polylactic acid may be a poly(L-lactic acid) resin, a poly(D-lactic acid) resin, or a poly(DL-lactic acid) resin. A blend of these resins may be used.

Examples of the other monomer that may be contained in the polylactic acid include aliphatic hydroxycarboxylic acids other than lactic acid, aliphatic polyhydric alcohols, aliphatic polycarboxylic acids, and polyfunctional polysaccharides.

When the polylactic acid is a copolymer of lactic acid and another monomer, it is preferable, in terms of the crystallinity of the polylactic acid, that the proportion of the other monomer in the total monomers contained in the polylactic acid be from about 0 to about 3 mol %. The proportion is more preferably from 0 to 2 mol %.

The lactic acid material used to produce the polylactic acid is not limited to a particular type; L-lactic acid, D-lactic acid, DL-lactic acid, or a mixture thereof may be used. Alternatively, L-lactide, D-lactide, meso-lactide, or a mixture thereof may also be used. Lactic acid obtained by microbial fermentation of a plant-derived renewable material such as starch can be suitably used.

The method for producing the polylactic acid is not limited to a particular technique and may be any known method such as dehydration polycondensation or ring-opening polymerization.

The molecular weight of the polylactic acid is not limited to a particular range, and may be set as appropriate depending on the intended purpose. The number-average molecular weight of the polylactic acid is preferably from 1,000 to 700,000 and more preferably from 10,000 to 300,000.

### (Copolyester)

The resin composition according to the first aspect of the present invention contains, in addition to the polylactic acid, a copolyester of lactic acid and another hydroxycarboxylic acid. The incorporation of this copolyester can improve the tensile elongation at break of the polylactic acid and reduce drawdown during melting.

By virtue of being a polyester containing lactic acid as a constituent monomer, the copolyester exhibits good compatibility with the polylactic acid and can form a homogeneous mixture with the polylactic acid. As a result, a substantial reduction in the transparency of the polylactic acid can be avoided.

The copolyester is itself a polymer material that exhibits biodegradability. Considering that bacteria capable of decomposing copolymers of lactic acid and 3-hydroxybutyric acid have been isolated from the environment (see Polymer Degradation and Stability, 2014, 110, 44; and Applied Microbiology and Biotechnology, 2015, 99, 9555), the copolyester is expected to exhibit high biodegradability in the environment, including in soil. In addition, enzymes secreted from the isolated copolyester-biodegrading bacteria have been isolated and found to decompose poly(D-lactic acid) oligomers up to a 31-mer.

The copolyester used has a high degree of copolymerization randomness in order to both improve tensile elongation at break and reduce drawdown. Specifically, a copolyester is used that satisfies the requirement that a copolymerization randomness, calculated as a ratio (b/a) of a theoretical value (b) of a triad content of the other hydroxycarboxylic acid in the copolyester to a measured value (a) of the triad content of the other hydroxycarboxylic acid, should be from 0.5 to 3.0. Within this range up to 3, the closer the ratio (b/a) is to 3, the higher the degree of copolymerization randomness.

The ratio (b/a) indicating the copolymerization randomness is preferably 0.7 or more, more preferably 0.8 or more, and even more preferably 0.9 or more. The ratio (b/a) may be up to 2.5, up to 2.0, up to 1.5, up to 1.2, or up to 1.1.

The use of the copolyester having such a high degree of copolymerization randomness can improve the tensile elongation at break of the resulting molded article and reduce drawdown during melting. If the copolymerization randomness is less than 0.5, it becomes difficult to achieve the improvement effect on tensile elongation at break, and excessive drawdown is likely to occur during melting.

As used herein, the term “triad content of the other hydroxycarboxylic acid” refers to the proportion of H-H-H triads each consisting of three linked units H of the other hydroxycarboxylic acid in the total amount of triads in which the central unit is the other hydroxycarboxylic acid. The “total amount of triads” includes H-H-H and other triads (L-H-H, L-H-L, and H-H-L) in which the lactic acid unit L is present adjacent to the central unit of the other hydroxycarboxylic acid.

The term “theoretical value (b) of a triad content of the other hydroxycarboxylic acid” refers to a value theoretically determined for the triad content of the other hydroxycarboxylic acid on the assumption that the lactic acid and the other hydroxycarboxylic acid are arranged completely at random in the copolyester. Such a theoretical value, which represents the proportion of triads in which both units adjacent to the central hydroxycarboxylic acid unit are also units of the other hydroxycarboxylic acid, is calculated as the square of the molar fraction of the other hydroxycarboxylic acid in the copolyester.

The measured value (a) of the triad content of the other hydroxycarboxylic acid can be calculated based on a spectrum obtained by ¹H-NMR spectroscopy of the copolyester. Specifically, the calculation can be made by taking advantage of the fact that signals derived from specific protons contained in the other hydroxycarboxylic acid are separated into signals attributed to the triad (H-H-H) of the other hydroxycarboxylic acid and signals attributed to other triads (L-H-H, L-H-L, and H-H-L). That is, the measured value (a) of the triad content of the other hydroxycarboxylic acid can be determined by calculating integrals of these signals.

When the copolyester is a block copolymer, the measured value (a) is nearly 100% or 1; thus, the ratio (b/a) is close to b. In contrast, when the lactic acid and the other hydroxycarboxylic acid are arranged in a perfectly regular pattern, the ratio (b/a) is greater than 1. As used herein, the phrase “arranged in a perfectly regular pattern” means that, for example, when the molar fraction of the lactic acid monomer units is 20 mol %, the lactic acid L and the other hydroxycarboxylic acid H are regularly arranged in a sequence such as -L-H-H-H-H-L-H-H-H-H-L-. In this case, since there are two H-H-H triads and two other triads L-H-H and H-H-L, the measured value (a) is 50%, and the theoretical value (b) is 64% (80%×80%). Accordingly, the ratio (b/a) is 1.28.

The lactic acid monomer units in the copolyester may be L-lactic acid monomer units or D-lactic acid monomer units. The lactic acid monomer units may include either or both L-lactic acid and D-lactic acid monomer units.

When the copolyester is microbially produced, the lactic acid monomer units in the copolyester consist essentially of D-lactic acid monomer units. As used herein, the phrase “consist essentially of D-lactic acid monomer units” means that the proportion of D-lactic acid monomer units in the total lactic acid monomer units is typically 90% or more, preferably 95% or more, and more preferably 99% or more.

The hydroxycarboxylic acid other than lactic acid that may be contained in the copolyester is not limited to a particular compound, and may be any hydroxycarboxylic acid copolymerizable with lactic acid. The number of carbon atoms in the hydroxycarboxylic acid is preferably 3 or more. The number of carbon atoms is preferably up to 15, more preferably up to 10, even more preferably up to 8, still more preferably up to 6, and particularly preferably up to 5.

The hydroxycarboxylic acid is preferably a hydroxyalkanoic acid. Specific examples of hydroxyalkanoic acids include 2-hydroxyalkanoic acids, 3-hydroxyalkanoic acids, and 4-hydroxyalkanoic acids. Among these, 3-hydroxyalkanoic acids are preferred.

Specific examples of 3-hydroxyalkanoic acids include 3-hydroxybutyric acid (hereinafter sometimes abbreviated as 3HB), 3-hydroxypentanoic acid, 3-hydroxyhexanoic acid, 3-hydroxyheptanoic acid, 3-hydroxyoctanoic acid, 3-hydroxynonanoic acid, 3-hydroxydecanoic acid, 3-hydroxydodecanoic acid, 3-hydroxytetradecanoic acid, 3-hydroxypentadecanoic acid, and 3-hydroxyhexadecanoic acid. One of these acids may be contained alone in the copolyester, or two or more may be contained in combination. In particular, the copolyester preferably contains at least 311B. Most preferably, the copolyester is P(LA-co-3HB) (hereinafter sometimes abbreviated as LAHB), which is a copolyester of lactic acid and 3HB.

The proportion of lactic acid monomer units in the copolyester is not limited to a particular range. However, in order to more effectively improve the tensile elongation at break of the polylactic acid and reduce drawdown during melting, the molar fraction of lactic acid monomer units in the total monomer units constituting the copolyester is preferably from 10 to 70 mol %, more preferably from 15 to 60 mol %, and even more preferably from 15 to 50 mol %. In order to particularly enhance the drawdown-reducing effect, the molar fraction is still more preferably up to 40 mol %, particularly preferably up to 30 mol %, and most preferably up to 25 mol %.

The molar fraction of lactic acid monomer units can be determined using HPLC. Alternatively, the molar fraction may be determined using NMR or GC.

The weight-average molecular weight Mw of the copolyester is set to be 11×10⁴ or more in order to ensure both improved tensile elongation at break and reduced drawdown. If the weight-average molecular weight of the copolyester is less than 11×10⁴, it becomes difficult to obtain the improvement effect on tensile elongation at break, and excessive drawdown is likely to occur during melting.

The weight-average molecular weight is preferably 13×10⁴ or more, more preferably 15×10⁴ or more, even more preferably 20×10⁴ or more, and particularly preferably 30×10⁴ or more. The upper limit is not limited to a particular value. In terms of productivity and melt processability, the weight-average molecular weight is preferably 300×10⁴ or less, more preferably 200×10⁴ or less, even more preferably 150×10⁴ or less, still more preferably 100×10⁴ or less, particularly preferably 80×10⁴ or less, and most preferably 60×10⁴ or less.

The weight-average molecular weight of the copolyester can be determined using a gel permeation chromatography (GPC) system (manufactured by Shimadzu Corporation) equipped with a Tandem TSKgel Super HZM-H column (manufactured by Tosoh Corporation) on a standard polystyrene basis.

The copolyester of lactic acid and the other hydroxycarboxylic acid can be produced using an organic resource of biological origin (biomass) other than fossil fuel. The copolyester can be produced from a material that is 100% biomass-derived.

The method used to produce the copolyester of lactic acid and the other hydroxycarboxylic acid is not limited to a particular technique, and may be any known method. The copolyester may be microbially biosynthesized or may be produced by chemical synthesis. In particular, examples of methods for producing P(LA-co-3HB) include production methods using genetically modified microorganisms, such as the methods described in WO 2009/131186 A1 and WO 2006/126796 A1.

Among genetically modified microorganisms, those belonging to the genus Cupriavidus have the ability to produce a copolyester having a high degree of randomness. This is because, when such microorganisms are cultured, the phase of cell growth is followed by a distinct phase of polymer biosynthesis, and a homogeneous polymer is continuously produced during the polymer biosynthesis. Accordingly, the use of a genetically modified microorganism belonging to the genus Cupriavidus facilitates production of the copolyester according to the first aspect of the present invention.

Alternatively, a genetically modified microorganism belonging to a genus other than the genus Cupriavidus (such as genetically modified Escherichia coli) may be used. In this case, the copolyester according to the first aspect of the present invention can be produced by regulating the aeration rate and the stirring speed during culturing.

The copolyester according to the first aspect of the present invention can be used as a soil biodegradation accelerator for accelerating soil biodegradation of polylactic acid. The soil biodegradation of polylactic acid can be accelerated by bringing the copolyester into contact with the polylactic acid. The details will be described later with respect to the second aspect.

### (Blend Proportions)

The blend proportions of polylactic acid and the copolyester in the resin composition according to the first aspect of the present invention may be set in view of improvement in tensile elongation at break and reduction of drawdown. The amount of the copolyester is preferably from 10 to 100 parts by weight per 100 parts by weight of the polylactic acid. In terms of improvement in tensile elongation at break, the amount is preferably up to 80 parts by weight, more preferably up to 60 parts by weight, and particularly preferably up to 50 parts by weight. The amount may be at least 15 parts by weight or at least 20 parts by weight.

### (Other Components)

The resin composition may contain a thermoplastic resin other than polylactic acid and the copolyester described above. Such an additional thermoplastic resin is not limited to a particular type, and any conventionally known resin may be used. Specific examples include biodegradable aliphatic polyesters other than polylactic acid and the copolyester, and aromatic polyesters.

The amount of the additional thermoplastic resin is not limited to a particular range. For example, the amount of the additional thermoplastic resin may be from 0 to 200 parts by weight per 100 parts by weight of the polylactic acid. This amount may be up to 100 parts by weight, up to 50 parts by weight, up to 30 parts by weight, up to 10 parts by weight, up to 5 parts by weight, up to 1 part by weight, or up to 0.1 parts by weight.

Resins having low compatibility with polylactic acid might deteriorate the transparency of the polylactic acid. Accordingly, it is preferable that such a resin not be incorporated, or be incorporated in a small amount. For example, although the following range is not limiting, the amount of polyhydroxyalkanoate resins, which are an example of resins having low compatibility with polylactic acid, is preferably from about 0 to about 100 parts by weight and more preferably from about 0 to about 50 parts by weight per 100 parts by weight of the polylactic acid.

The resin composition may contain other additives as appropriate, to the extent that the additives do not diminish the effect of the invention. Examples of such additives include, but are not limited to, plasticizers, hydrolysis inhibitors, compatibilizers, antioxidants, ultraviolet absorbers, processing aids, antistatic agents, colorants, nucleating agents, inorganic or organic particles, lubricants, mold release agents, water-repellent agents, inorganic fillers, antifungal agents, antimicrobial agents, blowing agents, and flame retardants. The amount of each additive may be chosen as appropriate depending on the intended purpose. One additive may be incorporated alone, or two or more additives may be incorporated in combination.

The plasticizer used may be one commonly used as a plasticizer for polymers. Specific examples include polyester plasticizers, glycerin plasticizers, polycarboxylate ester plasticizers, polyalkylene glycol plasticizers, and epoxy plasticizers.

### (Applications)

The resin composition according to the first aspect of the present invention can be processed into pellets by melting and kneading the components, extruding the resulting molten resin as a strand, and cutting the strand into pellets. The pellets thus obtained may be dried to remove moisture, and the dried pellets may be processed by a known molding method to obtain a molded article having any desired form. Such a molded article is also one embodiment of the present invention.

Examples of molding methods include film molding, sheet molding, injection molding, blow molding, fiber spinning, extrusion foaming, and bead foaming.

Examples of methods for producing film-molded articles include, but are not limited to, T-die extrusion molding, calender molding, roll molding, and blown film molding. The resulting film can be subjected to thermoforming using heating, vacuum molding, or press molding.

Examples of methods that can be employed to produce injection-molded articles include injection molding methods such as injection molding commonly used to mold thermoplastic resins, gas-assisted injection molding, and injection compression molding. Depending on the intended purpose, other methods may also be used, including in-mold injection molding, gas pressure injection molding, two-color molding, sandwich molding, push-pull injection molding, and SCORIM. The injection molding method to be used is not limited to those mentioned above.

The resin composition may be processed into pellets or into a film-shaped, sheet-shaped, or fiber-shaped molded article using an extrusion molding machine. Alternatively, the resin composition may be processed into a molded article having a given shape by injection molding.

When the resin composition contains a blowing agent, the molded article may be a foamable molded article or a molded foam obtained by foaming the foamable molded article.

The resin composition can be processed into various forms of molded articles. Examples of such molded articles include paper, films, sheets, tubes, plates, rods, containers, bags, and parts. These molded articles may be combined with another molded article (such as a fiber, a yarn, a rope, a woven fabric, a knit, a non-woven fabric, paper, a film, a sheet, a tube, a plate, a rod, a container, a bag, a part, or a foam) made of a material different from the resin composition according to the first aspect of the present invention.

The molded articles described above are not limited to particular applications, and are suitable for use in various fields such as agricultural industry, fishery industry, forestry industry, horticultural industry, medical industry, hygiene industry, apparel industry, non-apparel industry, packaging industry, automotive industry, building material industry, and other industries.

### [Second Aspect]

A soil biodegradation accelerator according to the second aspect of the present invention is used with polylactic acid to accelerate soil biodegradation of the polylactic acid. The soil biodegradation accelerator contains at least a copolyester of lactic acid and another hydroxycarboxylic acid.

### (Polylactic Acid)

As described above, polylactic acid is known to have low biodegradability in soil. However, biodegradation of polylactic acid in soil can be accelerated by using the soil biodegradation accelerator according to the second aspect of the present invention.

As used herein, the term “biodegradation” refers to a phenomenon in which the resin in question is decomposed into water and carbon dioxide through the action of microorganisms, and the term “soil biodegradation” refers to a phenomenon in which the resin in question undergoes biodegradation in soil.

With the use of the soil biodegradation accelerator according to the second aspect of the present invention, plastic materials containing polylactic acid as a main component can be composted not only by standard industrial composting methods but also by home composting or soil burial.

The polylactic acid in the second aspect will not be described in detail, as it is the same as the polylactic acid described with respect to the first aspect, except for the points described below.

### (Copolyester)

The soil biodegradation accelerator according to the second aspect of the present invention contains a copolyester of lactic acid and another hydroxycarboxylic acid as an active ingredient for accelerating soil biodegradation. When brought into contact with polylactic acid, the copolyester can accelerate soil biodegradation of the polylactic acid itself.

By virtue of being a polyester containing lactic acid as a constituent monomer, the copolyester exhibits good compatibility with polylactic acid and can form a homogeneous mixture with the polylactic acid. As a result, a substantial reduction in the transparency of the polylactic acid can be avoided. Furthermore, the copolyester can plasticize the polylactic acid, thereby increasing the elongation of the polylactic acid.

The copolyester according to the second aspect may be the same as that described with respect to the first aspect, or may be a copolyester of lactic acid and another hydroxycarboxylic acid that does not fall within the definition of the copolyester described with respect to the first aspect.

The copolyester according to the second aspect may be a random copolymer, an alternating copolymer, a block copolymer, or a graft copolymer. However, in terms of soil biodegradation-accelerating effect, transparency, mechanical properties, and availability, the copolyester is preferably a random copolymer. The term “random copolymer” refers to a copolymer in which two or more types of monomer units are arranged in a random sequence. When the copolyester is microbially produced, the copolyester is usually a random copolymer.

It should be understood that the copolyester used in the second aspect may or may not satisfy the requirement described with respect to the ratio indicating copolymerization randomness in the first aspect.

The lactic acid monomer units in the copolyester according to the second aspect may be L-lactic acid monomer units or D-lactic acid monomer units. The lactic acid monomer units may include either or both L-lactic acid and D-lactic acid monomer units.

When the copolyester according to the second aspect is microbially produced, the lactic acid monomer units in the copolyester consist essentially of D-lactic acid monomer units. The phrase “consist essentially of D-lactic acid monomer units” means that the proportion of D-lactic acid monomer units in the total lactic acid monomer units is typically 90% or more, preferably 95% or more, and more preferably 99% or more.

The definition and specific or preferred examples of the hydroxycarboxylic acid other than lactic acid that is contained in the copolyester according to the second aspect will not be described below, as they are the same as those described with respect to the other hydroxycarboxylic acid in the first aspect.

The proportion of lactic acid monomer units contained in the copolyester according to the second aspect is not limited to a particular range. However, in order to enhance the soil biodegradation-accelerating effect on polylactic acid, the molar fraction of lactic acid monomer units in the total monomer units constituting the copolyester is preferably from 10 to 70 mol %, more preferably from 15 to 60 mol %, and even more preferably from 15 to 50 mol %.

The molecular weight of the copolyester according to the second aspect is not limited to a particular range. The weight-average molecular weight Mw may be, for example, from 1×10⁴ to 100×10⁴, and is preferably from 1×10⁴ to 50×10⁴. The weight-average molecular weight of the copolyester according to the second aspect may be within the same range as the weight-average molecular weight of the copolyester described with respect to the first aspect. The weight-average molecular weight can be determined as previously described.

The method for producing the copolyester according to the second aspect is not limited to a particular technique, and may be any conventionally known method. Specific examples include those previously described.

The soil biodegradation accelerator according to the second aspect of the present invention may consist solely of the copolyester of lactic acid and another hydroxycarboxylic acid, or may further contain components other than the copolyester. Examples of such other components include known additives for resins. Specific examples include plasticizers, hydrolysis inhibitors, compatibilizers, antioxidants, ultraviolet absorbers, processing aids, antistatic agents, colorants, nucleating agents, inorganic or organic particles, lubricants, mold release agents, water-repellent agents, inorganic fillers, antifungal agents, antimicrobial agents, blowing agents, and flame retardants.

The amount of the copolyester in the soil biodegradation accelerator is not limited to a particular range, and may be, for example, from 10 to 100 wt %. The amount of the copolyester may be at least 30 wt %, at least 50 wt %, at least 70 wt %, at least 90 wt %, or at least 99 wt %.

### (Way of Use)

The soil biodegradation accelerator according to the second aspect of the present invention is used in such a manner that the accelerator is brought into contact with polylactic acid. As a result, the soil biodegradation-accelerating effect on polylactic acid can be obtained. Specifically, the soil biodegradation accelerator and polylactic acid are preferably mixed so that the two components come into contact with each other. It is particularly preferable that the two components be mixed uniformly, for example, by melt kneading or by mixing in an organic solvent followed by removal of the solvent.

### (Usage Amount)

The usage amount of the soil biodegradation accelerator according to the second aspect of the present invention may be any amount, provided that the use of the soil biodegradation accelerator can accelerate soil biodegradation of polylactic acid. In terms of the balance between the soil biodegradation-accelerating effect and the transparency or mechanical properties of polylactic acid, the soil biodegradation accelerator is preferably used in an amount such that the amount of the copolyester, which is an active ingredient of the soil biodegradation accelerator, is from 1 to 200 parts by weight per 100 parts by weight of the polylactic acid. The amount of the copolyester is more preferably from 5 to 100 parts by weight and even more preferably from 10 to 80 parts by weight. The amount of the copolyester may be at least 20 parts by weight or at least 30 parts by weight. The amount of the copolyester may be up to 60 parts by weight or up to 50 parts by weight.

The usage amount of the soil biodegradation accelerator may be within the same range as the amount of the copolyester per 100 parts by weight of polylactic acid in the first aspect.

### (Resin Composition)

The second aspect of the present invention may be a resin composition containing polylactic acid and the soil biodegradation accelerator described above. Because the incorporation of the soil biodegradation accelerator accelerates soil biodegradation of the polylactic acid, the resin composition can exhibit good soil biodegradability.

### (Other Components)

The resin composition according to the second aspect may contain a thermoplastic resin other than polylactic acid and the copolyester described above. Such an additional thermoplastic resin is not limited to a particular type, and any conventionally known resin may be used. Specific examples of the additional thermoplastic resin and its amount may be the same as those described with respect to the first aspect.

As in the first aspect, the resin composition according to the second aspect may contain other additives, to the extent that the additives do not diminish the effect of the invention.

### (Applications)

As in the first aspect, the resin composition according to the second aspect can be processed into pellets, and the pellets can be processed by a known molding method to obtain a molded article having any desired form. Such a molded article is also one embodiment of the present invention. The details and specific applications of the molded article will not be described below, as they are the same as those described with respect to the first aspect.

The molded article according to the second aspect exhibits good soil biodegradability. Accordingly, the molded article is particularly suitable for use in applications where the molded article may be composted through home composting or disposed of by soil burial. Specific examples include, but are not limited to, packaging materials, food packaging materials, cutlery goods, rubber bags, agricultural materials, and coated paper.

In the following items, preferred aspects of the present disclosure are listed. The present invention is not limited to the following items.

### [Item 1]

A resin composition containing:
polylactic acid; and
a copolyester of lactic acid and another hydroxycarboxylic acid, wherein
the copolyester has a weight-average molecular weight of 11×10⁴ or more, and
the copolyester has a copolymerization randomness of 0.5 to 3.0, the copolymerization randomness being calculated as a ratio (b/a) of a theoretical value (b) of a triad content of the other hydroxycarboxylic acid to a measured value (a) of the triad content of the other hydroxycarboxylic acid.

### [Item 2]

The resin composition according to item 1, wherein the other hydroxycarboxylic acid is a 3-hydroxyalkanoic acid.

### [Item 3]

The resin composition according to item 1 or 2, wherein the other hydroxycarboxylic acid is at least one hydroxycarboxylic acid selected from the group consisting of 3-hydroxybutyric acid, 3-hydroxypentanoic acid, 3-hydroxyhexanoic acid, 3-hydroxyheptanoic acid, 3-hydroxyoctanoic acid, 3-hydroxynonanoic acid, 3-hydroxydecanoic acid, 3-hydroxydodecanoic acid, 3-hydroxytetradecanoic acid, 3-hydroxypentadecanoic acid, and 3-hydroxyhexadecanoic acid.

### [Item 4]

The resin composition according to any one of items 1 to 3, wherein the other hydroxycarboxylic acid is 3-hydroxybutyric acid.

### [Item 5]

The resin composition according to any one of items 1 to 4, wherein lactic acid monomer units in the copolyester are D-lactic acid monomer units.

### [Item 6]

The resin composition according to any one of items 1 to 5, wherein a molar fraction of lactic acid monomer units in the copolyester is from 10 to 70 mol %.

### [Item 7]

The resin composition according to any one of items 1 to 6, wherein the weight-average molecular weight of the copolyester is from 20×10⁴ to 80×10⁴.

### [Item 8]

The resin composition according to any one of items 1 to 7, wherein the copolymerization randomness is from 0.8 to 3.0.

### [Item 9]

The resin composition according to any one of items 1 to 8, wherein an amount of the copolyester is from 10 to 100 parts by weight per 100 parts by weight of the polylactic acid.

### [Item 10]

The resin composition according to any one of items 1 to 9, wherein the copolyester functions as a soil biodegradation accelerator for accelerating soil biodegradation of the polylactic acid.

### [Item 11]

A molded article produced by molding the resin composition according to any one of items 1 to 10.

### [Item 12]

A soil biodegradation accelerator for accelerating soil biodegradation of polylactic acid, the soil biodegradation accelerator containing a copolyester of lactic acid and another hydroxycarboxylic acid, wherein
the copolyester has a weight-average molecular weight of 11×10⁴ or more, and
the copolyester has a copolymerization randomness of 0.5 to 3.0, the copolymerization randomness being calculated as a ratio (b/a) of a theoretical value (b) of a triad content of the other hydroxycarboxylic acid to a measured value (a) of the triad content of the other hydroxycarboxylic acid.

### [Item 13]

A method for accelerating soil biodegradation of polylactic acid, the method including bringing a copolyester of lactic acid and another hydroxycarboxylic acid into contact with the polylactic acid, wherein
the copolyester has a weight-average molecular weight of 11×10⁴ or more, and
the copolyester has a copolymerization randomness of 0.5 to 3.0, the copolymerization randomness being calculated as a ratio (b/a) of a theoretical value (b) of a triad content of the other hydroxycarboxylic acid to a measured value (a) of the triad content of the other hydroxycarboxylic acid.

### [Item 14]

Use of a copolyester of lactic acid and another hydroxycarboxylic acid as a soil biodegradation accelerator for accelerating soil biodegradation of polylactic acid, wherein
the copolyester has a weight-average molecular weight of 11×10⁴ or more, and
the copolyester has a copolymerization randomness of 0.5 to 3.0, the copolymerization randomness being calculated as a ratio (b/a) of a theoretical value (b) of a triad content of the other hydroxycarboxylic acid to a measured value (a) of the triad content of the other hydroxycarboxylic acid.

### [Item 15]

A soil biodegradation accelerator for accelerating soil biodegradation of polylactic acid, the soil biodegradation accelerator containing a copolyester of lactic acid and another hydroxycarboxylic acid.

### [Item 16]

A resin composition containing:
polylactic acid; and
the soil biodegradation accelerator according to item 15.

### [Item 17]

A method for accelerating soil biodegradation of polylactic acid, the method including bringing a copolyester of lactic acid and another hydroxycarboxylic acid into contact with the polylactic acid.

### [Item 18]

Use of a copolyester of lactic acid and another hydroxycarboxylic acid as a soil biodegradation accelerator for accelerating soil biodegradation of polylactic acid.

### EXAMPLES

Hereinafter, the present invention will be described in greater detail with reference to examples. It should be noted that the present invention is not limited to these examples. The overall genetic manipulations can be carried out, for example, as described in Molecular Cloning (Cold Spring Harbor Laboratory Press (1989)). The enzymes, cloning hosts, and the like used in the genetic manipulations can be purchased from market suppliers and used according to the instructions of the suppliers. The enzymes are not limited to particular species and may be any enzymes that can be used for the genetic manipulations.

### [Cultivation of Copolyester (LAHB)-Producing Hydrogen-Oxidizing Bacteria]

Cupriavidus necator H16 was genetically modified to prepare a strain KNK005ΔphaZ_{1,2,6}/nagE G793C.dR, in which: the PHA polymerase gene phaC_{1Re} on the genome was substituted with another PHA polymerase gene; the PHA depolymerase genes phaZ_{1,2,6} were disrupted; the 793rd base G of the nagE gene involved in N-acetylglucosamine uptake was replaced with C to enhance glucose-assimilating capacity; and the nagR gene encoding a transcriptional regulator was disrupted (see WO 2017/104722 A1).

The strain KNK005ΔphaZ_{1,2,6}/nagE G793C.dR was further modified to prepare H16 phaC_{1Re}::STQK ΔphaZ_{1,2,6}/nagE G793C.dR, in which the PHA polymerase gene phaC_{1Re} on the genome was replaced with an STQK mutant (a PHA polymerase obtained by mutating a Pseudomonas sp. 61-3-derived polymerase PhaC1_{Ps} through substitution of the 325th serine with threonine and the 481st glutamine with lysine). This strain is referred to as the “host (1).”

### (Preparation of Gene Disruption Plasmid)

PCR was performed using the genomic DNA of C. necator H16 as a template and the oligo-DNA sequences of SEQ ID NOS: 1 and 2 as primers. Prime STAR GXL DNA Polymerase (manufactured by Takara Bio Inc.) was used as the DNA polymerase. Similarly, PCR was carried out using the DNA sequences of SEQ ID NOS: 3 and 4 as primers. Overlap PCR was further performed using the two DNA fragments obtained by these PCR processes as templates and the DNA sequences of SEQ ID NOS: 3 and 4 as primers. The resulting DNA fragment was one in which about 500 base pairs upstream of the ORF of acetyl-CoA acetyltransferase (Locus tag: H16_A1438) and about 500 base pairs downstream of the ORF were linked together. This DNA fragment was treated with the restriction enzyme SmiI, and the resulting fragment was ligated, using a DNA ligase, to a vector pNS2X-sacB (described in Japanese Laid-Open Patent Application Publication No. 2007-259708) that had also been treated with SmiI. The resulting gene disruption plasmid containing the base sequence of SEQ ID NO: 5 was designated “pNS2X-sacB-ΔphaA.” This gene disruption plasmid is a plasmid used to disrupt the gene phaA:A1438 encoding acetyl-CoA acetyltransferase.

### (Preparation of Gene Introduction Plasmid)

A gene introduction plasmid was prepared to introduce genes required for LAHB synthesis into the genome of C. necator. Specifically, a plasmid pNS2X-sacB-phaJ4b::REP-LDH_{Lm} was prepared that was capable of introducing a gene sequence for expression of a Leuconostoc mesenteroides-derived lactate dehydrogenase LDH_{Lm} under the control of a REP promoter so as to replace the ORF of phaJ4b (Locus tag: H16_B0397) on the genome of the host (1). This plasmid was one prepared by inserting the DNA fragment of SEQ ID NO: 6 into the pNS2X-sacB vector through ligation. The use of this plasmid enables C. necator to produce D-lactic acid from glucose. This plasmid is referred to as the “gene introduction plasmid (1).”

Similarly, a plasmid pNS2X-sacB-phaJ4a::lacN17-PCT_{Es} was produced that was capable of introducing a gene sequence for expression of Epulopiscium sp.-derived propionyl-CoA transferase PCT_{Es} under the control of a lacN17 promoter so as to replace the ORF of phaJ4a (Locus tag: H16_A1070) on the genome of the host (1). This plasmid was one prepared by inserting the DNA fragment of SEQ ID NO: 7 into the pNS2X-sacB vector through ligation. The use of this plasmid allows addition of CoA to lactic acid produced from glucose, thereby enabling supply of a substrate for copolyester polymerase. This plasmid is referred to as the “gene introduction plasmid (2).”

(Genetic Alteration of C. necator by Homologous Recombination)

The gene disruption plasmid or gene introduction plasmid was introduced into Escherichia coli 517-1 (ATCC 47055) by electroporation, and the resulting strain was cocultured with the target genetically modified C. necator strain on Nutrient Agar (manufactured by Difco) for conjugation.

After coculturing, a strain carrying the plasmid inserted into the genome was selected from the cultured cells on a Simons agar medium (sodium citrate 2 g/L, sodium chloride 5 g/L, magnesium sulfate heptahydrate 0.2 g/L, diammonium hydrogen phosphate 1 g/L, agar 15 g/L, pH=6.8) containing 250 mg/L kanamycin sulfate, and the strain was isolated. The isolated strain was further purified on Nutrient Agar containing 250 mg/L kanamycin sulfate. The resulting strain was then inoculated into Nutrient Agar containing 15% sucrose to obtain strains from which the plasmid had been eliminated. Due to homologous recombination, two types of strains were generated during elimination of the plasmid: a strain that restored the original genome sequence, and a strain that underwent the intended genetic alteration. The latter strain was isolated by colony PCR. The genetically altered strain thus obtained was purified on Nutrient Agar containing sucrose. In this manner, a homologous recombination strain was obtained.

The host (1) was genetically altered by the procedures described above using the gene introduction plasmids (1) and (2) to prepare a strain H16 phaC_{1Re}::STQK ΔphaZ_{1,2,6}/nagE G793C.dR phaJ4a::lacN17-PCT_{Es} phaJ4b::REP-LDH_{Lm}. This strain is referred to as the “host (2).” This strain was further genetically altered by the procedures described above using the gene disruption plasmid to prepare a strain H16 phaC_{1Re}::STQK ΔphaZ_{1,2,6}/nagE G793C.dR phaJ4a::lacN17-PCT_{Es} phaJ4b::REP-LDH_{Lm}ΔphaA. This strain is referred to as the “host (3).”

A DNA fragment encoding STQK was amplified by PCR. The amplified fragment was ligated, using a DNA ligase, to a DNA fragment obtained by treating a pCUP2 vector (see WO 2007/049716 A1) with MunI and SpeI. This resulted in a vector for gene expression under the control of a strong lacUV5 promoter (SEQ ID NO: 8). This expression vector was introduced into the hosts (2) and (3) by electroporation to prepare strains, which are referred to as the “host (2′)” and “host (3′).” The plasmid was maintained by adding kanamycin as appropriate.

The hosts (2′) and (3′) were used to produce a copolyester (LAHB). This production was performed using a jar fermenter, with glucose as the carbon source.

First, preculturing was performed overnight at 30° C. using a meat medium (composition: 1% (w/v) meat extract, 1% (w/v) Bacto Tryptone, 0.2% (w/v) yeast extract, 0.9% (w/v) disodium hydrogen phosphate dodecahydrate, 0.15% (w/v) potassium dihydrogen phosphate, 50 g/L kanamycin).

The resulting preculture fluid was introduced into a 500-ml Sakaguchi flask containing 100 ml of a meat medium, and the flask contents were shake-cultured at 30° C. for 6 hours.

Subsequently, the resulting culture fluid was transferred to a 5-L jar fermenter (Bioneer Neo, manufactured by B.E. Marubishi Co., Ltd.) containing 1.8 L of a PHA production medium. The jar fermenter was operated at a culture temperature of 30° C., a stirring speed of 500 rpm, and an aeration rate of 1.8 L/min. This culturing was performed for 48 hours, during which the pH was controlled between 6.7 and 6.8. For the pH control, a 7% aqueous solution of ammonium hydroxide was used.

The PHA production medium had the following composition: 0.578% (w/v) disodium hydrogen phosphate dodecahydrate, 0.101% (w/v) potassium dihydrogen phosphate, 0.437% (w/v) ammonium sulfate, 0.15% (w/v) magnesium sulfate heptahydrate, and 0.75% (v/v) trace metal salt solution (a solution of 1.6% (w/v) iron(II) chloride hexahydrate, 1% (w/v) calcium chloride dihydrate, 0.02% (w/v) cobalt chloride hexahydrate, 0.016% (w/v) copper sulfate pentahydrate, and 0.012% (w/v) nickel chloride hexahydrate in 0.1 N hydrochloric acid). The carbon source was glucose, and its initial concentration was 20 g/L. After glucose was consumed to 10 g/L, the glucose concentration was maintained at 10 g/L.

Microbial cells were collected from the culture fluid by centrifugation, purified with pure water and ethanol, and dried by vacuum drying. The polymer was extracted from the dried cells with chloroform, and the chloroform was then thoroughly removed using an evaporator and a vacuum drier. As a result, a copolyester (LAHB) of lactic acid and 3-hydroxybutyric acid was obtained.

For the copolyester obtained from the host (2′), the molar fraction of lactic acid as measured by ¹H-NMR spectroscopy was 15 mol %, and the weight-average molecular weight was 54×10⁴. This copolyester is referred to as “HPL-1.”

For the copolyester obtained from the host (3′), the molar fraction of lactic acid as measured by ¹H-NMR spectroscopy was 20 mol %, and the weight-average molecular weight was 37×10⁴. This copolyester is referred to as “HPL-2.”

### (Synthesis Example 1) [Synthesis of ENL-1]

A copolyester of lactic acid and 3-hydroxybutyric acid was biosynthesized using genetically modified Escherichia coli in accordance with the teachings of PNAS 105 (45) 17323-17327 (2008). Culturing was performed at an aeration rate of 1 vvm and a stirring speed of 500 rpm, and the biosynthesized copolyester was extracted from the cultured cells with chloroform.

For the copolyester thus obtained, the molar fraction of lactic acid was 45 mol %, and the weight-average molecular weight Mw was 11.9×10⁴.

### (Synthesis Example 2) [Synthesis of ENL-2]

Culturing was performed as described above at an aeration rate of 2 vvm and a stirring speed of 500 rpm. For the copolyester thus obtained, the molar fraction of lactic acid was 22 mol %, and the weight-average molecular weight Mw was 7.6×10⁴.

### (Synthesis Example 3) [Synthesis of ENL-3]

Culturing was performed as described above at an aeration rate of 0.5 vvm and a stirring speed of 500 rpm. For the copolyester thus obtained, the molar fraction of lactic acid was 41 mol %, and the weight-average molecular weight Mw was 5.4×10⁴.

### (Synthesis Example 4) [Synthesis of ENL-4]

Culturing was performed as described above at an aeration rate of 0.5 vvm and a stirring speed of 300 rpm. For the copolyester thus obtained, the molar fraction of lactic acid was 54 mol %, and the weight-average molecular weight Mw was 7.3×10⁴.

### (Synthesis Example 5) [Synthesis of PHBH-1]

A copolyester of 3-hydroxyhexanoic acid and 3-hydroxybutyric acid was biosynthesized using genetically modified hydrogen-oxidizing bacteria in accordance with the teachings of WO 2015/115619 A1. The biosynthesized copolyester was extracted from the cultured cells with chloroform.

For the copolyester thus obtained, the molar fraction of 3-hydroxyhexanoic acid was 11 mol %, and the weight-average molecular weight Mw was 6.3×10⁴.

### [Calculation of Copolymerization Randomness]

A ¹H-NMR spectrum of each copolyester (LAHB) was measured using a nuclear magnetic resonance spectrometer (Avance III 600 MHz, manufactured by Bruker). The measurement was performed using deuterated chloroform as the solvent at room temperature with a scan number of 8.

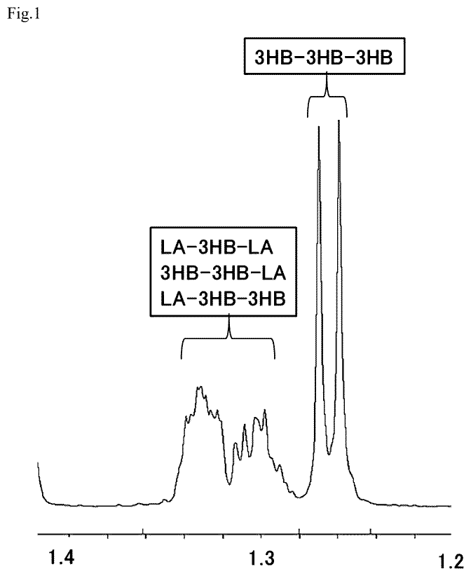

From the resulting spectrum, the area (X) of signals present in the region from 1.25 to 1.29 ppm, with tetramethylsilane set at 0 ppm, was determined, and the area (Y) of signals present in the region from 1.25 to 1.39 ppm was also determined. The measured value (a) of the triad content of 3-hydroxybutyric acid (3HB) was calculated as (X/Y)×100. The signals present in the region from 1.25 to 1.39 ppm are attributed to all methyl groups of 3-hydroxybutyric acid, and the signals present in the region from 1.25 to 1.29 ppm are attributed to the methyl groups contained in the triads (3HB-3HB-3HB) of 3-hydroxybutyric acid. Examples of these signals are shown in FIG. 1, which is an enlarged view of a region around 1.3 ppm in the ¹H-NMR spectrum.

When the monomer units are arranged completely at random, the theoretical value (b) of the 3HB triad content, which is the proportion of 3HB triads in the total 3HB units, can be determined as Z², where Z is the molar fraction of 3HB.

A copolymerization randomness, indicating how closely the monomer sequence of the copolyester approximates a completely random sequence, was determined as b/a. The results are shown in Table 1.

### Example 1

### [Measurement of Tensile Elongation at Break]

An amount of 10 g of a mixture containing 70 wt % polylactic acid (Ingeo 10361D, manufactured by NatureWorks) and 30 wt % copolyester (HPL-1) was dissolved in chloroform, and the solvent was removed to obtain a polymer blend sample as a dry solid. The obtained sample was molded into a 0.2-mm-thick film using a vacuum hot press at 180° C., and the film was punched to prepare a test piece having the shape of a JIS 5B-type tensile test specimen.

The test piece was measured for tensile elongation at break using a universal testing machine AG-IS (manufactured by Shimadzu Corporation) equipped with a thermostatic chamber. The measurement temperature was 22±1° C., and the test speed was 1 mm/min.

### [Evaluation of Drawdown Properties]

An amount of 10 g of a mixture containing 70 wt % polylactic acid (Ingeo 10361D, manufactured by NatureWorks) and 30 wt % copolyester (HPL-1) was melted, kneaded, and extruded using a twin-screw extruder (ULTNano05, manufactured by Technovel Corporation, screw diameter=1.5 cm, L/D=13.33) with the barrel and die set to 140° C. The molten strand discharged from the die having a diameter of 2.5 mm was pulled and transferred to a cooling step. The workability during this process was rated according to the following criteria, and used as an indicator of drawdown properties.

Excellent: The extruded molten strand had sufficient melt viscosity and tension, and was able to be pulled and transferred stably to the cooling step while maintaining a constant diameter.

Good: The extruded molten strand had moderate melt viscosity and tension, and it was possible to pull and transfer the strand to the cooling step by controlling the speed of transfer in accordance with the degree of sagging of the molten resin at the die exit.

Average: The extruded molten strand had low melt viscosity and tension, and the strand was barely able to be pulled and transferred continuously to the cooling step although the diameter of the strand varied during transfer.

Poor: Transfer of the extruded molten strand to the cooling step was difficult because the strand had excessively low melt viscosity and tension, and pulling the strand caused excessive extension and ultimate breakage of the strand.

### Examples 2 to 4

Polymer blend films were obtained and measured for tensile elongation at break, and drawdown properties were evaluated by melt extrusion. These procedures were performed in the same manner as in Example 1, except that HPL-2 was used as the copolyester and that the mixing proportions of polylactic acid and the copolyester were changed as shown in Table 1.

### Example 5

A polymer blend film was obtained and measured for tensile elongation at break, and drawdown properties were evaluated by melt extrusion. These procedures were performed in the same manner as in Example 1, except that ENL-1 was used as the copolyester and that the mixing proportions of polylactic acid and the copolyester were changed as shown in Table 1.

### Comparative Example 1

Tensile elongation at break was measured and drawdown properties were evaluated by melt extrusion in the same manner as in Example 1, except that polylactic acid (Ingeo 10361D, manufactured by NatureWorks) was used alone.

### Comparative Example 2

A polymer blend film was obtained and measured for tensile elongation at break, and drawdown properties were evaluated by melt extrusion. These procedures were performed in the same manner as in Example 1, except that PHBH-1 was used as the copolyester and that the mixing proportions of polylactic acid and the copolyester were changed as shown in Table 1.

### Comparative Example 3

A polymer blend film was obtained and measured for tensile elongation at break, and drawdown properties were evaluated by melt extrusion. These procedures were performed in the same manner as in Example 1, except that ENL-2 was used as the copolyester and that the mixing proportions of polylactic acid and the copolyester were changed as shown in Table 1.

### Comparative Example 4

A polymer blend film was obtained and measured for tensile elongation at break, and drawdown properties were evaluated by melt extrusion. These procedures were performed in the same manner as in Example 1, except that ENL-3 was used as the copolyester and that the mixing proportions of polylactic acid and the copolyester were changed as shown in Table 1.

### Comparative Examples 5 to 8

Polymer blend films were obtained and measured for tensile elongation at break, and drawdown properties were evaluated by melt extrusion. These procedures were performed in the same manner as in Example 1, except that ENL-4 was used as the copolyester and that the mixing proportions of polylactic acid and the copolyester were changed as shown in Table 1.

TABLE 1

| | Ex. 1 | Ex. 2 | Ex. 3 | Ex. 4 | Ex. 5 | Comp. 1 | Comp. 2 |
|---|---|---|---|---|---|---|---|
| Copolyester | HPL-1 | HPL-2 | HPL-2 | HPL-2 | ENL-1 | — | PHBH-1 |
| | | | | | | | (HH: 11 mol %) |
| LA fraction (mol %) | 15 | 20 | ← | ← | 45 | — | 0 |
| Mw | 540k | 370k | ← | ← | 119k | — | 63k |
| Measured value (a) of 3HB triad content | 71.6 | 63.9 | ← | ← | 43 | — | — |
| Theoretical value (b) of 3HB triad content | 72.9 | 63.8 | ← | ← | 30.3 | — | — |
| Copolymerization randomness (b/a) | 1.02 | 1.00 | ← | ← | 0.70 | — | — |
| PLA proportion (wt %) | 70 | 80 | 70 | 50 | 82 | 100 | 80 |
| | | | | | | (PLA alone) | |
| Copolyester proportion (wt %) | 30 | 20 | 30 | 50 | 18 | 0 | 20 |
| Tensile elongation at break (%) | 165 | 168 | 123 | 125 | 212 | 11 | 63 |
| Drawdown properties | Excellent | Excellent | Excellent | Excellent | Good | Poor | Poor |

| | Comp. 3 | Comp. 4 | Comp. 5 | Comp. 6 | Comp. 7 | Comp. 8 |
|---|---|---|---|---|---|---|
| Copolyester | ENL-2 | ENL-3 | ENL-4 | ENL-4 | ENL-4 | ENL-4 |
| LA fraction (mol %) | 22 | 41 | 54 | ← | ← | ← |
| Mw | 76k | 54k | 73k | ← | ← | ← |
| Measured value (a) of 3HB triad content | 62 | 44 | 55 | ← | ← | ← |
| Theoretical value (b) of 3HB triad content | 60.8 | 34.8 | 21.2 | ← | ← | ← |
| Copolymerization randomness (b/a) | 0.98 | 0.79 | 0.39 | ← | ← | ← |
| PLA proportion (wt %) | 70 | 80 | 90 | 80 | 70 | 50 |
| Copolyester proportion (wt %) | 30 | 20 | 10 | 20 | 30 | 50 |
| Tensile elongation at break (%) | 67 | 8 | 7 | 14 | 24 | 20 |
| Drawdown properties | Average | Poor | Average | Average | Average | Average |

Table 1 reveals that in Examples 1 to 5, in which polylactic acid was blended with a copolyester having a weight-average molecular weight Mw of 11×10⁴ or more and a copolymerization randomness of 0.5 to 3.0, the tensile elongation at break was markedly increased and the drawdown properties were also improved, as compared with Comparative Example 1 in which polylactic acid was used alone.

In Comparative Example 2, in which the copolyester used was PHBH-1 containing no lactic acid, the tensile elongation at break was somewhat increased but remained unsatisfactory, and the drawdown properties were not improved. In Comparative Examples 3 to 8, which did not satisfy either or both the requirements concerning Mw and copolymerization randomness, the tensile elongation at break and the drawdown properties were not sufficiently improved.

### [Preparation of Test Samples]

Polylactic acid (Ingeo 2003D, manufactured by NatureWorks; D-lactic acid content=4 mol %) and a copolyester (HPL-2) were melted and kneaded at a weight ratio of 60:40 or 70:30. The resulting mixture was then frozen and pulverized into a powder having an average particle size adjusted to about 500 m. The powders thus obtained were used as test samples in Examples 6 and 7. Cellulose (Cellulose microcrystalline, manufactured by Merck KGaA) was used as a reference sample (Reference Example 1).

### [Home Composting Test]

A home composting test was conducted according to JIS K 6953-2. For evaluation of biodegradability (degradability in soil), a compost was prepared by mixing a plant source (100 g of soil, aged at 28° C. for 14 days), sea sand (85 g), and water (15 g). The resulting compost was placed into a glass container and then mixed with 7 g of the test sample or cellulose as a reference sample and 7 g of water. The mixture was maintained at 28° C. The amount of carbon dioxide evolved was measured, and the amount of carbon dioxide evolved in the absence of the test sample or cellulose was subtracted. The resulting value was divided by the theoretical amount of carbon dioxide evolved, and the quotient was defined as the degree of biodegradation. Water was added to the compost at weekly intervals to maintain the initial weight of the compost. The results are shown in Table 2.

TABLE 2
[Results of home composting test]

| | | Copolyester amount | Degree of biodegradation (%) | | | |
|---|---|---|---|---|---|---|
| | Sample | (wt %) | 124th day | 129th day | 150th day | 180th day |
| Ex. 6 | Polylactic acid + | 40 | 42 | 43 | 47 | 48 |
| | copolyester | | | | | |
| Ex. 7 | Polylactic acid + | 30 | 29 | 30 | 32 | 34 |
| | copolyester | | | | | |
| Ref. 1 | Cellulose | — | 98 | 100 | — | — |

As a result of measurement, it was confirmed that biodegradation proceeded well in Reference Example 1 in which cellulose was used.

In Example 6 in which the test sample was used, the degree of biodegradation was 42% when calculated on the assumption that only the copolyester was completely biodegraded into carbon dioxide. In Example 7, the degree of biodegradation thus determined was 32%. If the degree of biodegradation exceeds these values, this means that a component other than the copolyester, that is, polylactic acid, also undergoes biodegradation.

As a result of measurement, it was confirmed that on the 124th day in Example 6 and on the 150th day in Example 7, the degree of biodegradation exceeded the level calculated on the assumption that only the copolyester was completely biodegraded. This means that not only the copolyester but also polylactic acid was biodegraded. These results reveal that the presence of the copolyester accelerated biodegradation of polylactic acid in the compost.

The degree of biodegradation on the 180th day reached 48% in Example 6 and 34% in Example 7.

### DISCUSSION

The blend of the copolyester and polylactic acid exhibits high transparency. This fact leads to the inference that the two components are homogeneously mixed or that they form a morphology in which islands of the copolyester are finely dispersed in a matrix of polylactic acid at a scale smaller than the wavelength of light.

In the former case, the plasticizing effect of the copolyester is expected to facilitate movement of molecular chains of the polylactic acid, thereby enabling degrading enzymes to readily access the polylactic acid and allowing water molecules to easily penetrate into the polylactic acid. This is a possible reason why the accelerated biodegradation of polylactic acid was observed.

In the latter case, the copolyester islands exposed on the surface of the blend are decomposed through the action of copolyester-degrading enzymes to leave voids, and considerably fine asperities are formed on the surface of the polylactic acid, whereby the surface area of the polylactic acid can be markedly increased. As a result, the polylactic acid undergoes chemical hydrolysis to produce polylactic acid oligomers that can be enzymatically degraded. This is a possible reason why the accelerated biodegradation of polylactic acid was observed. In this case, it can also be expected that low-molecular-weight compounds produced by decomposition of the copolyester remain in the voids of the polylactic acid and act as acids to accelerate hydrolysis on the surface of the polylactic acid.

In addition, when the copolyester is present on the surface of the blend, microorganisms capable of secreting enzymes involved in hydrolysis of the copolyester and assimilating the resulting decomposition products preferentially grow. It can also be considered that the presence of a high concentration of hydrolases produced by such microorganisms accelerated hydrolysis of the polylactic acid.

It is inferred that the accelerating effect on soil biodegradation of polylactic acid was achieved by any one of the mechanisms described above or by any combination of two or more thereof.

### Example 8

### [Preparation of Compound Pellets]

An amount of 10 g of a mixture containing 97 wt % polylactic acid (Ingeo 2003D, manufactured by NatureWorks) and 3 wt % copolyester (HPL-2) was dried at 50° C. for 12 hours, after which the dried mixture was melted, kneaded, and extruded using a twin-screw extruder (ULTNano05, manufactured by Technovel Corporation, screw diameter=1.5 cm, L/D=13.33) with the barrel and die set to 190° C. The molten strand discharged from the die having a diameter of 2.5 mm was pulled and transferred to a cooling step, in which the strand was air-cooled. The strand was then cut using a pelletizer into pieces having a length of about 4 mm. In this manner, compound pellets were obtained.

### Comparative Example 9

Polylactic acid pellets were obtained in the same manner as the pellets of Example 8, except that 10 g of polylactic acid (Ingeo 2003D, manufactured by NatureWorks) was used alone.

The compound pellets obtained in Example 8 or the pellets obtained in Comparative Example 9 were dried using a vacuum dryer (DP300, manufactured by Yamato Scientific Co., Ltd.) at 80° C. under vacuum for 4 hours. Subsequently, the dried pellets were processed into a 4-mm-thick press sheet using a hydraulic vacuum hot press (IMC-11FD, manufactured by Imoto Machinery Co., Ltd.) at a molding pressure of 1.4 MPa and a temperature of 170° C. under vacuum.

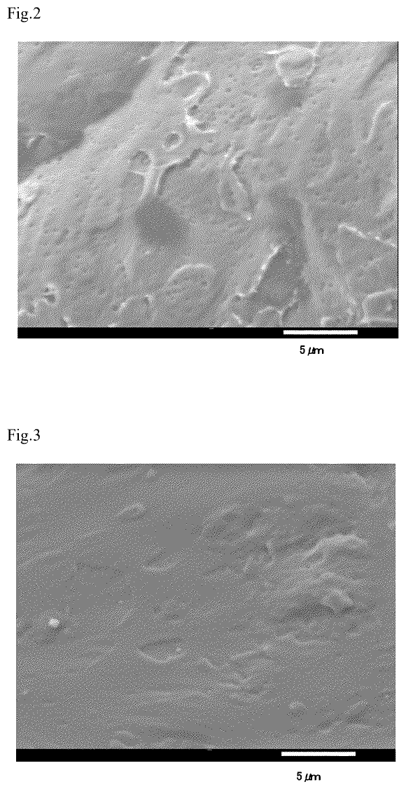

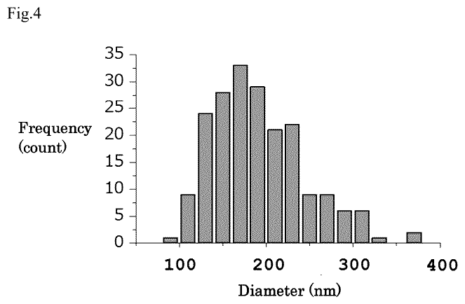

The press sheet thus obtained was frozen using liquid nitrogen and then fractured. The resulting fractured surface was observed with a scanning electron microscope (SEM; JSM-IT 300 HR, manufactured by JEOL Ltd.). Such SEM images are shown in FIGS. 2 and 3.

FIG. 2 shows the test piece of Example 8, on the surface of which a large number of circular concave features were observed. These concave features were formed because the copolyester of 3-hydroxybutyric acid and lactic acid was very finely dispersed in polylactic acid.

In contrast, in the test piece of Comparative Example 9 shown in FIG. 3, no such features were observed.

For the SEM observation result shown in FIG. 2, the diameters of 200 circular concave features were measured using image analysis software, ImageJ. A histogram showing the diameter distribution thus obtained is shown in FIG. 4. The diameters were generally distributed over a range of 100 to 350 nm, and the mean diameter was 185±5 nm.

1-16. (canceled)

17. A method for accelerating soil biodegradation of polylactic acid, the method comprising bringing a copolyester of lactic acid and another hydroxycarboxylic acid into contact with the polylactic acid.

18. (canceled)

19. The method according to claim 17, wherein the copolyester has a weight-average molecular weight of at least 11×10⁴, and the copolyester has a copolymerization randomness of 0.5 to 3.0, the copolymerization randomness being calculated as a ratio (b/a) of a theoretical value (b) of a triad content of the other hydroxycarboxylic acid to a measured value (a) of the triad content of the other hydroxycarboxylic acid.

20. The method according to claim 17, wherein the other hydroxycarboxylic acid is a 3-hydroxyalkanoic acid.

21. The method according to claim 17, wherein the other hydroxycarboxylic acid is at least one hydroxycarboxylic acid selected from the group consisting of 3-hydroxybutyric acid, 3-hydroxypentanoic acid, 3-hydroxyhexanoic acid, 3-hydroxyheptanoic acid, 3-hydroxyoctanoic acid, 3-hydroxynonanoic acid, 3-hydroxydecanoic acid, 3-hydroxydodecanoic acid, 3-hydroxytetradecanoic acid, 3-hydroxypentadecanoic acid, and 3-hydroxyhexadecanoic acid.

22. The method according to claim 17, wherein the other hydroxycarboxylic acid is 3-hydroxybutyric acid.

23. The method according to claim 17 wherein lactic acid monomer units in the copolyester are D-lactic acid monomer units.

24. The method according to claim 17, wherein a molar fraction of lactic acid monomer units in the copolyester is from 10 to 70 mol %.

25. The method according to claim 19, wherein the weight-average molecular weight of the copolyester is from 20×10⁴ to 80×10⁴.

26. The method according to claim 19, wherein the copolymerization randomness is from 0.8 to 3.0.

27. The method according to claim 17, wherein an amount of the copolyester is from 10 to 100 parts by weight per 100 parts by weight of the polylactic acid.


SEQUENCE LISTING (8 sequences)

SEQ ID NO: 1 - DNA, 30 bp, synthetic construct
  source 1..30: mol_type = other DNA
tccatttaaatcgaagtgagcggcacgccg

SEQ ID NO: 2 - DNA, 31 bp, synthetic construct
  source 1..31: mol_type = other DNA
accccttccttaacgttcatgcacatacgtg

SEQ ID NO: 3 - DNA, 29 bp, synthetic construct
  source 1..29: mol_type = other DNA
atgaacgttaaggaaggggttttccgggg

SEQ ID NO: 4 - DNA, 31 bp, synthetic construct
  source 1..31: mol_type = other DNA
cccatttaaattggtgtcgatcaccgcatcc

SEQ ID NO: 5 - DNA, 824 bp, Cupriavidus necator
  source 1..824: mol_type = other DNA
atttaaatcgaagtgagcggcacgccgatcgacctcaaacaggtcactgccgacatctactccctggccggcaccaacgatcacatcacgccctggaagtcttgctacaagtcggcgcaactgttcggtggcaaggtcgaattcgtgctgtccagcagtgggcatatcaagagcattctgaacccgccgggcaatccgaaatcacgttacatgaccagcaccgacatgccagccaccgccaacgagtggcaagaaaactcaaccaagcacaccgactcctggtggctgcactggcaggcctggcaggccgagcgctcgggcaaactgaaaaagtccccgaccagcctgggcaacaaggcctatccgtcaggagaagccgcgccgggcacgtatgtgcatgaacgttaaggaaggggttttccggggccgcgcgcggttggcgcggacccggcgacgataacgaagccaatcaaggagtggacatgactcagcgcattgcgtatgtgaccggcggcatgggtggtatcggaaccgccatttgccagcggctggccaaggatggctttcgtgtggtggccggttgcggccccaactcgccgcgccgcgaaaagtggctggagcagcagaaggccctgggcttcgatttcattgcctcggaaggcaatgtggctgactgggactcgaccaagaccgcattcgacaaggtcaagtccgaggtcggcgaggttgatgtgctgatcaacaacgccggtatcacccgcgacgtggtgttccgcaagatgacccgcgccgactgggatgcggtgatcgacaccaatttaaat

SEQ ID NO: 6 - DNA, 2781 bp, synthetic construct
  source 1..2781: mol_type = other DNA
atttaaataggccatcgagatcacggcggagtacctgaaggtgcgcaagcagttcggcgtggcgatcggcagcttccagtcgctgcagcaccgggtgtcggacatggccgccgagatggaagtcgcccgctcgatgctttacgtgctgctggcagccatcgaaaacgacagccccgacctggaccgcaccgtgtcgcaggccaagagcctggtggtgcgcgccgcgcgctttgtgtgcggacaggccatccagctgcacggcggcattggcatgaccgaggaatatccagtcggccattacttcaagcgcgccgtcgtggcggacgcgctgttcggcaatgccgacatgcacgatgcgcgcaatgccgaggcatggcaggcatccctgactgaataggaagagcaagccaattccccgggcaagtaccttgccgacatctatgcgctggcgcgcacgcgcctggcgcgcgccggctgtaccgaggtctacggcggcgacgcctgcaccgtggccgacgccggtcgcttctactcctatcggcgcgatggcgtgaccggccgcatggccagcctggtctggctggcggactgagcccgccgctgcctcactcgtccttgcccctggccgcctgcgcgcgctcggcttcagccttgcgtcggcggcggccgggcgtgcccatgatgtagagcaccagcgccaccggcgccatgccatacatcaggaaggtggcaacgcctgccaccacgttgtgctcggtgatcgccatcatcagcgccacgtagagccagccaatggccacgatgtacatcaaaaattcatccttctcgcctatgctctggggcctcggcagatgcgagcgctgcataccgtccggtaggtcgggaagcgtgcagtgccgaggcggattcccgcattgacagcgcgtgcgttgcaaggcaacaatggactcaaatgtctcggaatcgctgacgattcccaggtttctccggcaagcatagcgcatggcgtctccatgcgagaatgtcgcgcttgccggataaaaggggagccgctatcggaatggacgcaagccacggccgcagcaggtgcggtcgagggcttccagccagttccagggcagatgtgccggcagaccctcccgctttgggggaggcgcaagccgggtccattcggatagcatctccccatgcaaagtgccggccagggcaatgcccggagccggttcgaatagtgacgcaattgcacgtgcagagagacaatcaaatcatgaagatcttcgcctacggcatccgcgacgacgagaagccgtcgctggaggaatggaaggccgccaacccggagatcgaagtggactacacccaggagctgctgaccccggaaaccgtgaagctggccgagggcagcgactcggccgtggtgtaccagcagctggactacacccgcgaaaccctgaccgccctggccaacgtgggcgtgaccaacctgagcctgcgcaacgtgggcaccgacaacatcgacttcgacgccgcccgcgagttcaacttcaacatctcgaacgtgccggtgtacagcccgaacgccatcgccgaacactcgatgatccagctgtcgcgcctgctgcgtcgcaccaaggccctggacgccaagatcgccaagcacgacctgcgctgggccccgaccatcggccgcgagatgcgcatgcagaccgtgggcgtgatcggcaccggccacatcggccgcgtggccatcaacatcctgaagggcttcggcgccaaggtgatcgcctacgacaagtacccgaacgccgagctgcaggccgaaggcctgtacgtggacaccctggacgaactgtacgcccaggccgacgccatcagcctgtacgtgccgggcgtgccggagaaccaccacctgatcaacgccgaagccatcgccaagatgaaggacggcgtggtgatcatgaacgccgcccgcggcaacctgatggacatcgacgccatcatcgacggcctgaactcgggcaagatcagcgacttcggcatggacgtgtacgagaacgaagtgggcctgttcaacgaggactggtcgggcaaggaattcccggacgccaagatcgccgacctgatcagccgcgagaacgtgctggtgaccccgcacaccgccttctacaccaccaaggccgtgctggaaatggtgcaccagagcttcgacgccgccgtggccttcgccaagggcgagaagccggccatcgccgtggaatactgacctgccggcctggttcaaccagtcggcagccgactagttttccataaggcgccgcacgccgcgcggtgacgacgacgggcgggtctgctggcaggccgcccgtcttttgccattcaggcaccccggcagacagaccgcccccacggcagtgttgccgtctagccaaccaccgtcgttcccgcgcatacccgaagggcacgtgggcgggaacccagtgactttgatcccgaaggggacgtgaagacgctggattcccgcctgcgcgggaatgacggtagcgcttgatgccttaacaggactacgtcatccccatgcaggggactcaatagaaggaggagacaggcatgaccagaccccattcgacacgccgccgccacatcctggcgctggccgccagcagcgtcatccagacgctggcaccggccgcgcgggcgcaggcgcgcggctacccggaccgcccgctcaggatcgtgtccgtcgcgagcccgggatcgggcatcgacgactacaatttaaat

SEQ ID NO: 7 - DNA, 2679 bp, synthetic construct
  source 1..2679: mol_type = other DNA
atttaaattatttcgaggactttgaagtcggcagccgccgcgaactgggttcgtacctcgtcaccgaggaagagatcctggcctttgcccgccagtacgatccgcagccgttccatatcgacaaggaagccgctgccaggagcatctatggcggcgtgatctcgagcggctggatgacgtgctcgatcatgatgcggctgctggtgctgagcaccaccggcaagtcggccagcatgggctcgcctggcgtggacgagatccgctggctcaagcccgtctacgccggcgacacgctcaccgtcgtgctgaatgtgctcgatacccggccgtcgcagtccaagccggaccgcggcgtggtccacacccagtgggaagccaccaaccagcgcggcgaactggtctgcaccgtcaagggcatgggcatgtacggccgccgccccgcttaagaacactttatcgtcaagaggagactatcgaccaattcgcgcaacgcaattaatgtgagttagctcactcattaggcaccccaggctttacacttatgcttccggctcgtatgttgtgtggaattgtgagcggataacaatttcacacaggaaacaattgcacgtgcagagagacaatcaaatcatgcgcaaagtcaaggtgatcacggcggacgaagccgcgatgctgatcaaggatggcgacaccgtgacgacctcggggttcgtggcctccgcgattccggaagccctgaacaaggccgtcgagaagcgctttctggaaaccgggagccccaagaacatcacgctgacctactgcggttcgcagggcaaccgcgatggccgcggcgcggagcactgggcccacaaggggctgctgaagcggttcatcgccggccattgggccaccgtcccgaagctgggccagatggccctgaataacgaaatcgaagcctacaacgtgagccaaggcgccctgtgccacctgtatcgcgacatcgcggcgcacaagccgggttgcttcaccaaggtcggcctgggcaccttcatcgatccgcgcaatggcggcggcaaggtgaacgacatcacgaaggaggacatcgtggaactggtcacgatcaaaggcgaagagtacctgttctatcccgccttcccgatcaacgtggcgatgatccgcggtacctacgcagacgagtccggcaacattacctttgagaaggaggtggccccgctggagggcaccagcgtcgcacaggcggtgaagaacagcggcggcatcgtgatcgtccaggtcgagcgcatcgtgaaggccggcaccctggacccgcgcctggtgaaagtgcccggcatctacgtggactatgtcgtggtggcagacccggaggaccatcgccagtcgctggactgcgattacgacccgacgctgtcgggcgaaaagcgcgcgccggacgtcctgcccgagccgctgcccctgagcgccaagaagatcatcggccgtcgcggcgcgctggaactggaaaaggacgtggccgtgaacctcggcgtgggtgcaccggaatacgtcgcctcggtggcgtcggaagaggggatcggcgactacatgacgctgaccgtcgagggcggcccggtgggcggcctgccccagggcggcatccgtttcggcgcctcgatgaatccggatgccgtgatcgaccagggttaccagttcgacttctacgacggcggcggtctggatctggcgtacctcggcctggcggagtgcgacgagaagggcaacatcaacgtgtcgcgcttcggccccaagatcgccggctgcggcggcttcatcaacatcacccagaataccccgaaggtgttcttctgcggcacctttacggccggcggcctgaaggtgaaggtcgaggacggcaaggtcatcatcgcccaagaagggcgcgagaagaagttcatcaagaaggtcgggcagatcaccttcaacggcgacgtggcgctgaagaacaagcaacaggtgatgtatatcaccgaacgctgcgtgttcgaactgaaggaagatggcctgcacctgaccgaaatcgcgccgggcatcgacctccagacgcagattctcgacgtcatggagttcgagccgaagattgaccgggtgaacggcgagatcaaactcatggacgcccggctgttctccgaggccctcatgggcctcaagtgacctgccggcctggttcaaccagtcggcagccgactagtcggatacgcagttttgacgcggtgcgacaatatgccacacaggcatgtcgcgcccctggaggccgcctgatgcgcggctttctgcatttttaggaaccgatttgtgacgttgatgcctgtcaaatgctgcttcgcggatttgtgcgtcgattgtccgttctgctgaacagtgtgttctgtgccgttgggtttacccctaatgttgcgttgcggtacagtcgcgcccacgatgttttatgtgttgcgctccccggccgaaaaccgcgcttgccatgaaggcaagtccgtcgggaagtgcaaaaacctcgtattccaacgctacccggccgccgtactgagccgggaccgttcgcggtttcggccaggtgccgcagcgccggcaaggtcctacagggcctgcagcgcacgcagtcagttgtccttccgtgttcatggcgttgcaaatttaaat

SEQ ID NO: 8 - DNA, 147 bp, synthetic construct
  source 1..147: mol_type = other DNA
gcgcaacgcaattaatgtgagttagctcactcattaggcaccccaggctttacactttatgcttccggctcgtataatgtgtggaattgtgagcggataacaatttcacacaggaaacaattgcacgtgcagagagacaatcaaatc